(19) United States
(12) Patent Application Publication
(10) Pub. No.: US 2004/0122456 A1
(43) Pub. Date: Jun. 24, 2004

(54) Methods and apparatus for gastric reduction

(76) Inventors: Vahid C. Saadat, Saratoga, CA (US); Richard C. Ewers, Fullerton, CA (US); Eugene Chen, Carlsbad, CA (US)

Correspondence Address: Nicola A. Pisano, Esq., Luce, Forward, Hamilton & Scripps, Suite 200, 11988 El Camino Real, San Diego, CA, 92130, US

(21) Appl. No.: 10/612,170

(22) Filed: Jul. 1, 2003

Related U.S. Application Data
(60) Provisional application No. 60/433,065, filed on Dec. 11, 2002.

(51) Int. Cl. A61B 17/08

(52) U.S. Cl. 606/157

(57) ABSTRACT

Apparatus for use with a gastric reduction system for narrowing a cross-sectional area of a patient's gastro-intestinal lumen. The apparatus comprises an anchor that is adapted to be reconfigured from a reduced delivery profile within a delivery catheter to an expanded deployed profile once the anchor is released from the delivery catheter.

### REFERENCE TO RELATED APPLICATIONS

This application claims priority from U.S. Provisional Patent Application No. 60/433,065, filed Dec. 11, 2002, which is incorporated herein by reference in its entirety.

### FIELD OF THE INVENTION

The present invention relates to methods and apparatus for reducing the effective cross-sectional area of a gastro-intestinal (“GI”) lumen.

### BACKGROUND OF THE INVENTION

Morbid obesity is a serious medical condition pervasive in the United States and other countries. Its complications include hypertension, diabetes, coronary artery disease, stroke, congestive heart failure, multiple orthopedic problems and pulmonary insufficiency with markedly decreased life expectancy.

Several surgical techniques have been developed to treat morbid obesity, e.g., bypassing an absorptive surface of the small intestine, or reducing the stomach size. These procedures are difficult to perform in morbidly obese patients because it is often difficult to gain access to the digestive organs. In particular, the layers of fat encountered in morbidly obese patients make difficult direct exposure of the digestive organs with a wound retractor, and standard laparoscopic trocars may be of inadequate length.

In addition, previously known open surgical procedures may present numerous life-threatening post-operative complications, and may cause atypical diarrhea, electrolytic imbalance, unpredictable weight loss and reflux of nutritious chyme proximal to the site of the anastamosis. Further, the sutures or staples that are often used in these surgical procedures may require extensive training by the clinician to achieve competent use, and may concentrate significant force over a small surface area of the tissue, thereby potentially causing the suture or staple to tear through the tissue.

In view of the aforementioned limitations, it would be desirable to provide methods and apparatus for achieving gastric reduction by reconfiguring the GI lumen of a patient.

It also would be desirable to provide methods and apparatus for gastric reduction using anchors that can be reconfigured from a reduced delivery profile to an expanded deployed profile.

It further would be desirable to provide methods and apparatus for gastric reduction wherein the anchors include atraumatic endpoints to minimize trauma to the patient's GI lumen.

It further would be desirable to provide methods and apparatus for gastric reduction wherein anchors are biased to an expanded deployed profile so that the anchors automatically deploy when released from a delivery catheter.

### SUMMARY OF THE INVENTION

In view of the foregoing, it is an object of the present invention to provide methods and apparatus for achieving gastric reduction by approximating tissue to reconfigure the GI lumen of a patient.

It is another object of the present invention to provide methods and apparatus for gastric reduction using anchors that can be reconfigured from a reduced delivery profile to an expanded deployed profile.

It is an additional object of this invention to provide methods and apparatus for gastric reduction wherein the anchors include atraumatic endpoints to minimize trauma to the patient's GI lumen.

It is a further object of the present invention to provide methods and apparatus for gastric reduction wherein anchors are biased to an expanded deployed profile so that the anchors automatically deploy when released from a delivery catheter.

These and other aspects of the present invention are accomplished by providing a gastric reduction system including methods and apparatus for delivering a plurality of anchors on opposing sides of a gastro-intestinal lumen and then moving the anchors to approximate the opposing walls of the lumen. In accordance with the principles of the present invention, the anchors may have any of a variety of configurations employing radially expanding sleeves or struts.

One aspect of the present invention involves using anchors to narrow a cross-sectional area of a gastro-intestinal lumen. The anchors each comprise a sleeve including proximal and distal bushings, wherein the sleeve is configured to transition between a reduced delivery profile and an expanded deployed profile. The anchor further comprises at least one suture coupled to the distal bushing and extending through the interior of the sleeve and an aperture in the proximal bushing. Application of tension on the suture pulls the distal bushing towards the proximal bushing, causing the sleeve to expand radially outward to the expanded deployed profile.

The sleeve may comprise a braided polymeric material or shape-memory alloy. Alternatively, the sleeve may comprise a hollow cylinder having longitudinal slots disposed through its wall to form a plurality of longitudinal struts that bow outward when the anchor is deployed. Optionally, the sleeve also may comprise a filament to facilitate later removal of the anchor or an internal locking mechanism, such as a ferrule and a corresponding barb, for retaining the sleeve in the expanded deployed profile. A coating of bioactive agent also may be applied to an outer surface of the sleeve to either promote or hinder tissue ingrowth.

In a further alternative embodiment, the anchor may comprise a plurality of struts having proximal and distal ends, a proximal bushing coupled to the proximal ends of the struts, and a plurality of central loops, wherein each central loop couples the distal ends of a pair of opposing struts. The central loops act as torsion springs that bias the anchor to the expanded deployed profile. Optionally, at least one suture may be attached to the central loop.

According to another embodiment, the anchor comprises a plurality of longitudinal struts coupled to proximal and distal bushings, so that the struts form a petaled disk-like configuration when deployed.

In still further embodiments, the anchor comprises a plurality of self-expanding struts hinged to a distal bushing, and a membrane that extends over the struts to facilitate self-expansion of the anchor. The ends of the struts opposite the distal bushing may be twisted into small loops to provide substantially atraumatic end points, or a central shank may be provided to form a fixture point for attachment of a suture. The distal bushing optionally may include a sharpened distal end to facilitate tissue penetration. As yet another alternative embodiment, the struts may be disposed within a windowed tube so that the windows act as stops to limit the radial expansion of the struts in the deployed state.

In still further embodiments, the anchor comprises a plurality of struts disposed substantially within a slotted tube that permits radial rotational expansion of the struts, wherein each strut is attached at one end to a coil spring disposed against an inner wall of the slotted tube. The slotted tube is dimensioned to receive an obturator such that insertion of the obturator through the slotted tube compresses the coil springs forcing the struts to rotate from the expanded deployed profile to the reduced delivery profile.

An anchor constructed in accordance with the present invention further may include an elongate shaft carrying fluid expandable elements at a distal end of the elongate shaft.

### BRIEF DESCRIPTION OF THE DRAWINGS

The above and other objects and advantages of the present invention will be apparent upon consideration of the following detailed description, taken in conjunction with the accompanying drawings, in which like reference characters refer to like parts throughout, and in which:

FIG. 1 is a schematic view of an illustrative delivery catheter for use with the gastric reduction methods of the present invention;

FIG. 2 is a side-sectional view of the delivery catheter of FIG. 1, loaded with an anchor of the present invention, penetrating a GI tissue wall of a patient;

FIG. 3 is a perspective view of the handle of the catheter of FIGS. 1 and 2;

FIGS. 4A and 4B are views of one preferred embodiment of an anchor of the present invention in the reduced delivery state;

FIGS. 5A-5C are side views depicting transmural implantation of the anchor assembly of FIGS. 4A-4B;

FIG. 6 is a perspective view of a fastener suitable for use with the anchors of the present invention;

FIGS. 7A-7E are cross-sectional views depicting methods of using the gastric reduction system of the present invention;

FIG. 8 is a side view of an alternative anchor;

FIGS. 9A and 9B are, respectively, side views of a wire malecot anchor according to the present invention in a reduced delivery profile and expanded deployed profile;

FIGS. 10A and 10B are, respectively, side views of an alternative wire malecot anchor of the present invention in a reduced delivery profile and expanded deployed profile;

FIGS. 11A and 11B are, respectively, side views of another alternative wire malecot anchor of the present invention in a reduced delivery profile and expanded deployed profile;

FIGS. 12A and 12B are, respectively, a side-sectional view off another anchor of the present invention disposed within a delivery catheter and in the deployed profile, while FIG. 12C is an alternative embodiment of the anchor of FIG. 12A;

FIGS. 13A-13C are, respectively, side-sectional views of another alternative anchor disposed within a delivery catheter in a reduced delivery profile and showing deployment of the anchor, while FIG. 13D is an end view of the deployed anchor;

FIGS. 14A and 14B are, respectively, side and side-sectional views of further alternative anchors having a slotted tube; while FIG. 14C is a side-sectional view of the anchor disposed within a delivery catheter;

FIGS. 15A and 15B are, respectively, side and side-sectional views of another alternative anchor, while FIG. 15C is a side-sectional view of the anchor disposed within a delivery catheter;

FIGS. 16A and 16B are, respectively, an end view of a further anchor of the present invention in an expanded deployed state and disposed within a delivery catheter;

FIGS. 17A and 17B are, respectively, side views of another anchor in a reduced delivery state and expanded deployed state;

FIGS. 18A and 18B are, respectively, side views of yet another anchor in a reduced delivery state and expanded deployed state;

FIGS. 19A and 19B are, respectively, side views of a still further anchor in a reduced delivery state and expanded deployed state;

FIGS. 20A and 20B are, respectively, a perspective view of a further anchor of the present invention in an expanded deployed state and disposed within a delivery catheter;

FIGS. 21A and 21B are, respectively, a perspective view of an anchor for use with an obturator and a side-sectional view of the anchor within a delivery catheter;

FIGS. 22A to 22F are various side views of alternative anchors having spider-like configurations;

FIGS. 23A and 23B are side views of wire anchors in an expanded delivery state according to the present invention;

FIG. 24 is a side-sectional view of an anchor including an internal lock according to the present invention; and

FIG. 25 is a side view of an anchor including a coating of bioactive agent according to the present invention.

### DETAILED DESCRIPTION OF THE INVENTION

Overview of a Preferred Gastric Reduction System

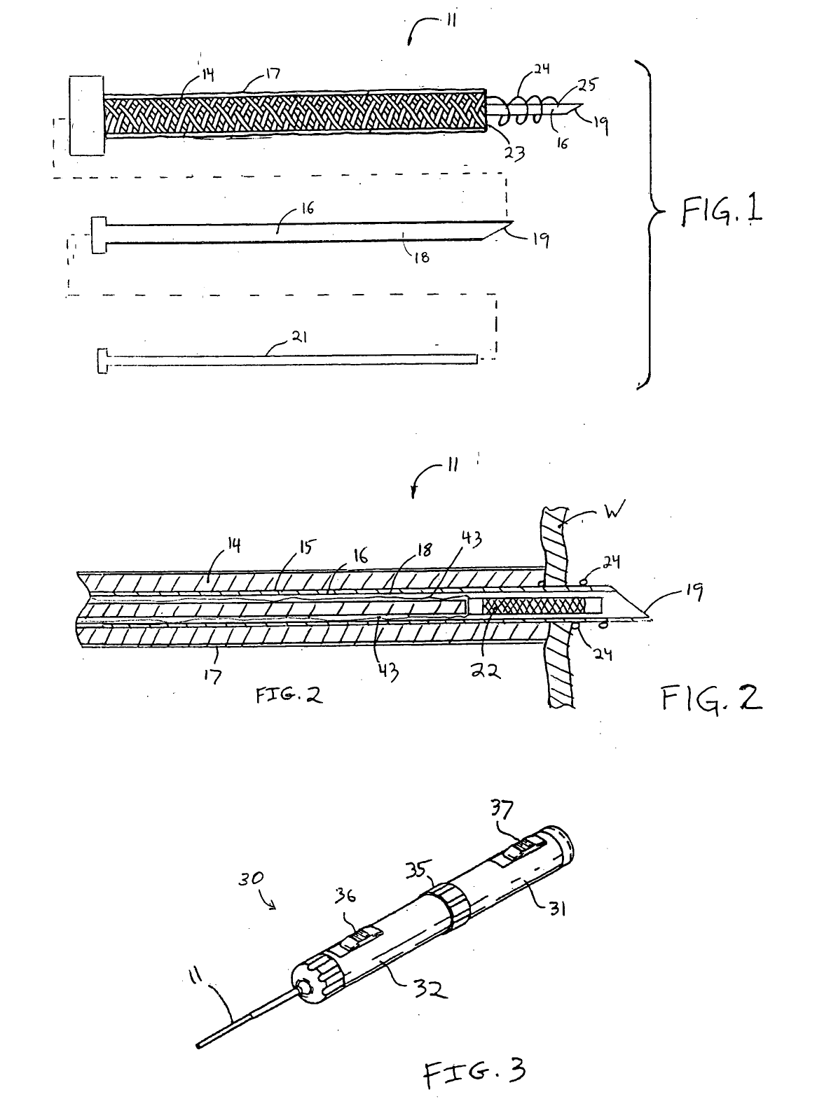

Referring to FIGS. 1-7, illustrative components of gastric reduction apparatus 10 in accordance with the principles of the present invention are described. As explained in detail hereinafter, apparatus 10 enables a clinician to treat obesity by approximating the walls of a gastro-intestinal lumen to narrow the lumen, thus reducing the area for absorption in the stomach or intestines. Gastric reduction system 10 comprises anchor delivery catheter 11, anchor 22, and optionally, suture tensioning assembly 50. The structure and operation of each of these components are described separately below.

A. Delivery Catheter

Referring now to FIGS. 1 and 2, an illustrative embodiment of delivery catheter 11 constructed in accordance with the principles of the present invention is described. Delivery catheter 11 comprises elongate torqueable tube 14 having lumen 15 and needle 16 disposed for translation within lumen 15. Torqueable tube 14 preferably is formed of braided stainless steel wire having TEFLON coating 17. Needle 16 includes lumen 18 and non-coring distal tip 19 that facilitates penetration of tissue wall W. Needle 16 preferably is configured to penetrate tissue wall W so that the tissue anchor, described below, may employ a substantially atraumatic distal tip.

Push rod 21 is disposed for translation within lumen 18, and is configured to eject anchor 22 (see FIG. 2) out of distal end 23 of the delivery catheter and through tissue wall W. As shown in FIG. 2, one or more sutures 43 are attached to anchor 22, and extend through lumen 18 of needle 16 so that the proximal ends of the sutures 43 extend out of the mouth of the patient.

To facilitate penetration of needle 16 into tissue wall W, delivery catheter 11 preferably includes coil 24 that may be engaged to tissue wall W to stabilize distal end 23 of delivery catheter 11 against the tissue during actuation of needle 16. Coil 24 preferably is attached at one end to distal end 23 of catheter 11 and terminates at the other end in sharpened tip 25. Coil 24 defines a central passage that permits needle 16 to be reciprocated therethrough.

Referring to FIG. 3, an illustrative handle 30 for controlling operation of delivery catheter 11 is described. Handle 30 comprises proximal portion 31 and distal portion 32. Distal portion 32 is coupled to elongate tube 14 so that rotation of knob 35 rotates coil 24 to engage wall W of the gastro-intestinal tissue, as illustrated in FIG. 2. Handle 30 further comprises slider buttons 36 and 37 for imparting translational movement to needle 16 and push rod 21, respectively.

In operation, after knob 35 has been rotated to engage coil 24 to tissue wall W, slider button 36 is actuated to urge needle 16 distally to pass through coil 24 and penetrate wall W. Once needle tip 19 has penetrated the tissue wall, slider button 37 is actuated to urge push rod 21 distally, thus ejecting anchor 22 from needle 16 on the distal side of tissue wall W. After the anchor assembly has been deployed, slider buttons 36 and 37 are retracted in the proximal direction to retract the needle and push rod back within elongate tube 14. Knob 35 may then be rotated in the opposite direction to release its engagement with tissue wall W.

B. Anchor

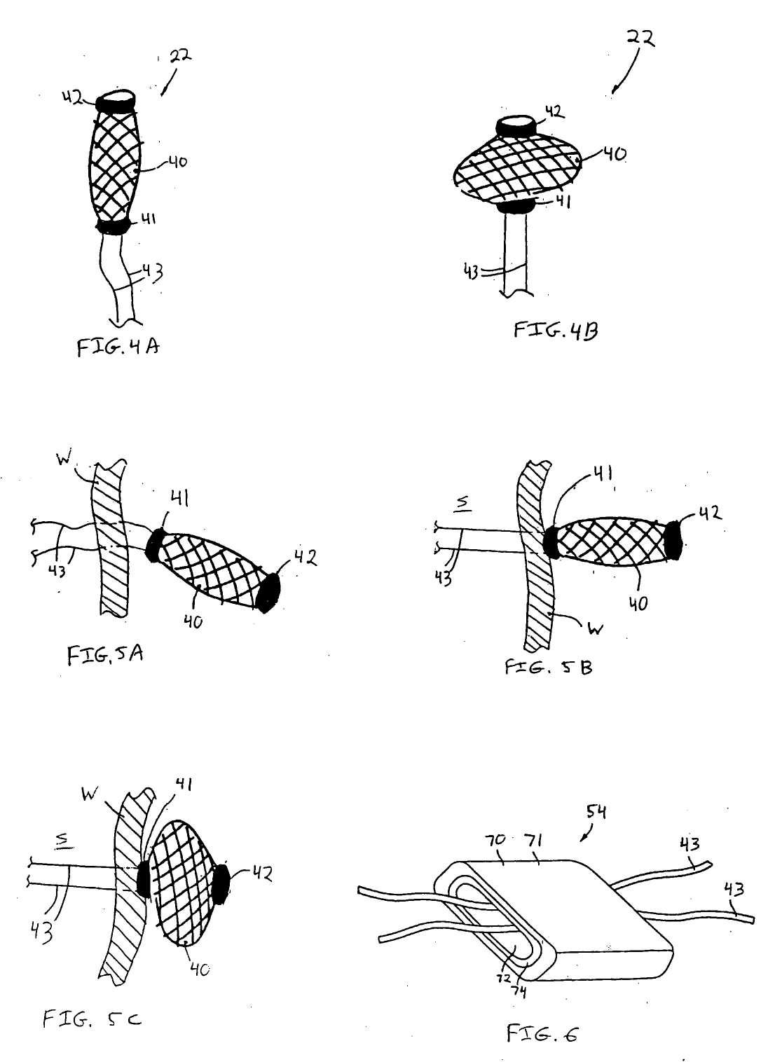

Referring now to FIGS. 4A and 4B, a preferred embodiment of anchor 22 constructed in accordance with the principles of the present invention is described. Anchor 22 comprises braided sleeve 40 coupled to proximal bushing 41 and distal bushing 42. One or more sutures 43 are coupled to distal bushing 42 and extend through bushing 41. Proximal bushing 41 may slide along the suture(s) relative to the distal bushing 42, so that braided sleeve expands radially outward. Accordingly, after anchor 22 is disposed through a tissue wall (as depicted in FIG. 2), application of tension to the sutures causes the anchor to transition from an elongate reduced delivery profile (FIG. 4a) to an expanded, substantially disk-shaped deployed profile (FIG. 4B).

Braided sleeve 40 preferably comprises a highly porous, compliant and high strength material composed of numerous individual monofilament elements. Suitable materials for the monofilament elements include polyester, nylon, TEFLON, polypropylene and combinations thereof. Braided sleeve 40 also may be formed from a shape memory metal, such as a Nickel-Titanium alloy. In addition, the porous braid structure may promote an easily and uniformly absorbable structure for use in applications in which anchor 22 is not intended for permanent implantation. Conversely, the porous braid structure may promote tissue growth to enhance anchoring in applications in which anchor 22 is designed for permanent implantation.

Anchor 22 may be made by thermo-forming two ends of a short length of braided sleeve to form proximal and distal bushings 41 and 42. Alternatively, separate bushings may be glued, over-molded, soldered or welded onto the ends of a length of braided sleeve. Suture(s) 43 may be attached to distal bushing 42 at a fixture point comprising, for example, one or more holes 46 formed in the distal bushing. Alternatively, the sutures may be attached using an eyelet, adhesive or other suitable fastener.

FIGS. 5A-5C depict deployment of anchor 22 from the reduced delivery profile to the expanded deployed profile. In FIG. 5A, anchor 22 has been forced through tissue wall W, illustratively the stomach wall, via needle lumen 18. Once delivery catheter 11 is withdrawn, anchor 22 is left disposed through tissue wall W with untensioned sutures 43 extending into the patient's stomach S. Sutures 43 pass through the esophagus and extend from the patient's mouth where they may be manipulated by the clinician.

In FIG. 5B, sutures 43 are shown partially tensioned, so that proximal bushing 41 engages the distal surface of tissue wall W. Because the stomach wall comprises a tough, resilient material, contact between the expanded braided sleeve and distal surface of the tissue wall causes the braided sleeve to partially expand, rather than slip back into the stomach via the track left by needle 16. When further tension is applied to sutures 43, distal bushing 42 is approximated toward proximal bushing 41, thereby causing braided sleeve 40 to expand in the radially to the substantially disk-shaped profile shown in FIG. 5C.

Alternatively, anchor 22 may be preformed to self-expand to disk-shaped profile to automatically upon ejection from lumen 18 of needle 16. Such a preset shape may be accomplished by coupling the anchor to a fixture (e.g., a mandrel) and heat setting the braided sleeve in the disk-shaped profile. For example, the bushings may be approximated and then retained in close proximity by a fixture, or the shape may be imposed by compressing the braid in a disk-shaped mold. The formed anchor and fixture then may be placed into an oven for a predetermined amount of time, and quenched or slowly cooled to room temperature.

C. Suture Tensioning Assembly

Referring now to FIG. 6, illustrative suture fastener 54 constructed in accordance with the principles of the present invention is described. Fastener 54 comprises collar 70 having body 71 and channel 72 through which sutures 43 may freely translate prior to crimping. Once fastener 54 is crimped, sutures 43 are restrained from further translation through channel 72, thus retaining a desired amount of tension on sutures 43. Optionally, body 71 may incorporate lining 74 to enhance friction between body 71 and suture 43, thereby reducing the risk of slippage.

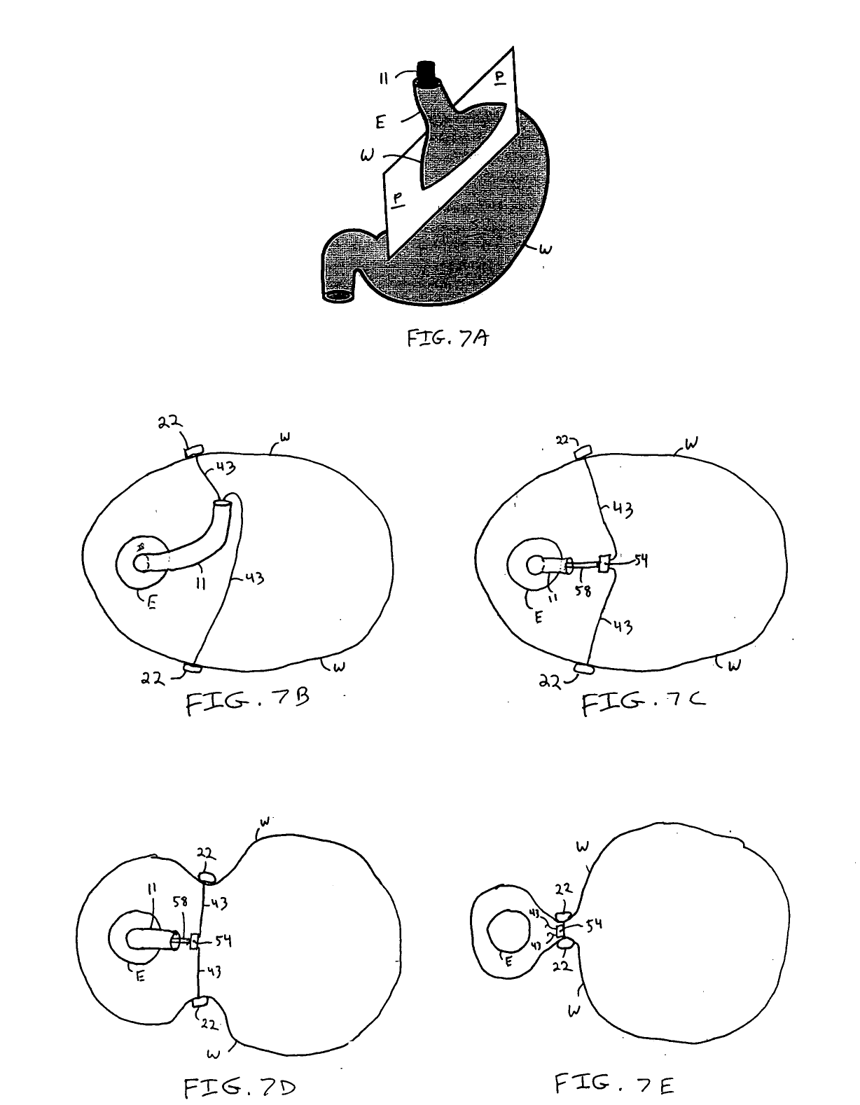

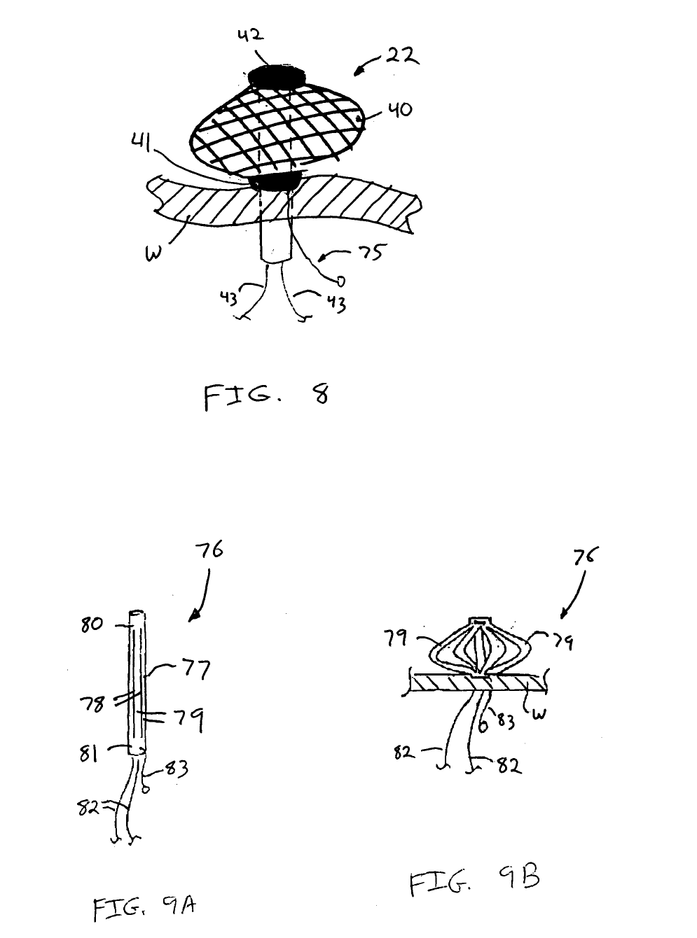

FIGS. 7A to 7E illustrate the steps of one procedure using gastric reduction system 10 to treat obesity. In FIG. 7A delivery catheter 11 of FIGS. 1-3 is inserted through a patient's mouth, esophagus E and stomach S. FIGS. 7B-7E depict cross-sectional views of the stomach taken along plane P of FIG. 7A.

FIG. 7B depicts a step in the which a pair of anchors 22 have been positioned through opposing tissue walls W of the stomach so that sutures 43 pass from each anchor through esophagus E and extend out of the patient's mouth. FIG. 7C depicts a step in which sutures 43 have been threaded through the channel of fastener 54. At this point, fastener 54 has not been crimped and may be freely translated along sutures 43 using a push rod. More particularly, tension is maintained in the sutures while push rod 58 is used to urge fastener 54 through patient's mouth and esophagus E and into the stomach.

FIG. 7D depicts a step in which fastener 54 is moved to a position approximately midway between anchors 22. Push rod 58 then is used to hold the fastener in place while additional tension is applied to the sutures, thereby causing opposing walls W of the stomach to bow inward toward one another. As depicted in FIG. 7E, the application of additional tension pulls the opposing tissue walls into proximity with each other, thereby narrowing the cross-sectional area of stomach S.

At this step in the procedure, fastener 54 is crimped to maintain the tension in sutures 43. The excess length of sutures 43 is cut and removed via the patient's mouth. Advantageously, narrowing of stomach S limits the amount of food the patient consumes by providing a feeling of satiety after only a small amount of food is ingested.

Alternatively or in addition, sutures 43 may comprise self-tightening materials that shrink over time, or materials such as nickel titanium or electroactive polymers that are pre-stretched so that the subsequent application of heat or electricity causes the sutures to shorten. By way of example, if pre-stretched nickel titanium or electroactive polymeric sutures are used, heat from a radiofrequency device or hot water may be used after the procedure to induce the sutures to tighten. Tension may be controlled by the ability of the sutures to tighten to a specific load. Tension also may be maintained by tying a knot or fusing the sutures to each other via application of heat.

Alternative Anchor Embodiments Suitable For Use With The Gastric Reduction System

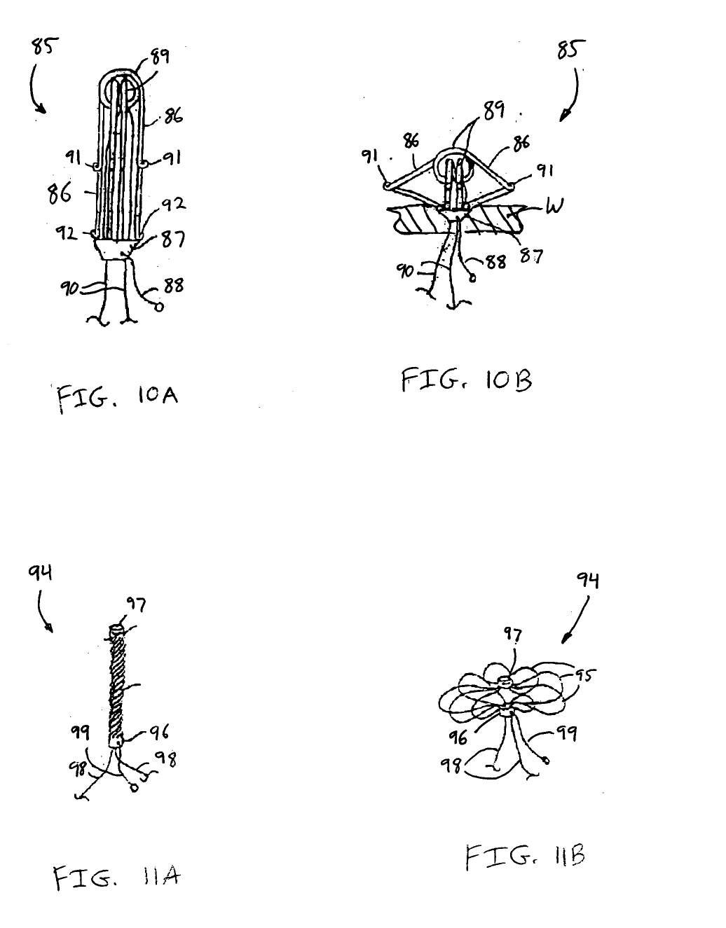

Referring to FIG. 8, mesh anchor 22 of the present invention includes secondary filament 75 coupled to proximal bushing 41. The application of tension on secondary filament 75 pulls the proximal bushing through the tissue wall. As the anchor is pulled through the wall, it resumes the elongate, reduced delivery profile. Advantageously, this permits the anchor to be selectively removed from a tissue wall, e.g., at completion of a predetermined course of treatment.

Alternatively, braided sleeves 40 of the embodiment of FIGS. 4 may be replaced by expandable malecot structures. FIGS. 9A-9B depict wire malecot anchor 76 formed, for example, from tube 77 having a plurality of longitudinal through-wall slots 78 to create struts 79. Preferably, the unslotted ends of tube 77 form distal and proximal bushings 80 and 81.

Wire malecot anchor 76 also includes one or more sutures 82 attached to distal bushing 80. When tension is applied to sutures 82, struts 79 bow radially outward to deploy the anchor to an expanded disk-like configuration (FIG. 9B). In addition, wire malecot anchor 76 also may include secondary filament 83 that permits the anchor 76 to be retrieved through the tissue wall at conclusion of a treatment. Wire malecot anchor 76 may be delivered through tissue wall W using delivery catheter 11 of FIGS. 1-3 to perform the procedure depicted in FIGS. 7A-7E.

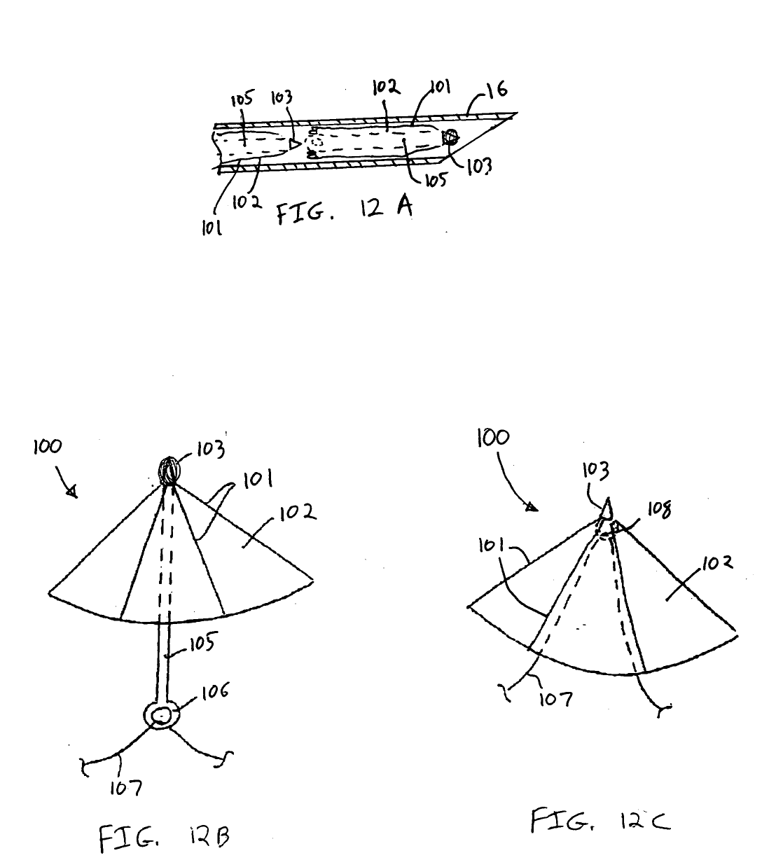

With respect to FIGS. 10A and 10B, an alternative embodiment of a malecot anchor is described. Spring wire malecot anchor 85 includes plurality of struts 86 coupled at the proximal end to proximal bushing 87. Proximal bushing 87 also may include secondary filament 88 to facilitate retrieval of the anchor through tissue wall W. Struts 86 may be formed, for example, by plastically deforming a continuous length of polymeric or metal wire around a mandrel.

Each strut 86 is coupled at its distal end to an opposing strut via loop 89. Loops 89 form a fixture point for one or more sutures 90. Preferably, as shown in FIG. 10A, each pair of opposing struts has first flexure point 91 substantially midway between loop 89 and the proximal bushing and second flexure point 92 disposed adjacent the proximal bushing. Flexure points 91 and 92 facilitate transition of the anchor between the reduced deliver profile and the expanded deployed profile.

In operation, loops 89 act as torsion springs that bias the anchors in the expanded deployed configuration of FIG. 10B. Advantageously, loops 89 allow the struts to withstand greater stresses before additional plastic deformation or failure of the struts. This increased capacity also facilitates self-expansion of the spring wire malecot anchor from the reduced delivery profile to the expanded deployed profile.

Spring wire malecot anchors 85 may be delivered through the tissue wall of a patient using a delivery catheter 11 such as disclosed in FIGS. 1-3. More particularly, when anchors 85 are disposed in delivery catheter 11, the delivery catheter radially constrains the anchors in the reduced delivery profile so that the struts are aligned with the longitudinal axis of the catheter. When an anchor is deployed, the radial constraint imposed by the catheter is removed, thereby permitting the anchor to self-expand into the expanded profile, wherein each strut 86 bows radially outward. Expansion of the deployed anchor is further reinforced when sutures 90 are tensioned.

With respect to FIGS. 11A and 11B, petaled malecot anchor 94 is described. Petaled malecot anchor 94 includes plurality of struts 95 coupled to proximal and distal bushings 96 and 97, respectively. In addition, one or more sutures 98 are attached to distal bushing 97. Petaled malecot anchor 94 also may include secondary filament 99 to facilitate retrieval of the anchor. In the expanded deployed profile, struts 95 form a petaled disk-like configuration, as depicted in FIG. 11B.

Petaled malecot anchor 94 may be formed by cutting angled slots into a cylindrical tube. Alternatively, the petaled structure may be created by joining the ends of thin longitudinal struts, so that a petaled structure results when the struts are compressed. Alternatively, a plurality of thin struts may be attached at either end to bushings. By way of example, a suitable material for use in constructing petaled malecot anchor 94 is nitinol wire.

Advantageously, the spiral structure of the petaled malecot anchor provides greater surface area contact with the tissue wall. In addition, the spiral structure includes few, if any, sharp angles and is therefore relatively atraumatic. Moreover, the struts of the petaled malecot naturally take the form of a loop in the expanded deployed configuration, and do not have stress concentration points that may be susceptible to failure.

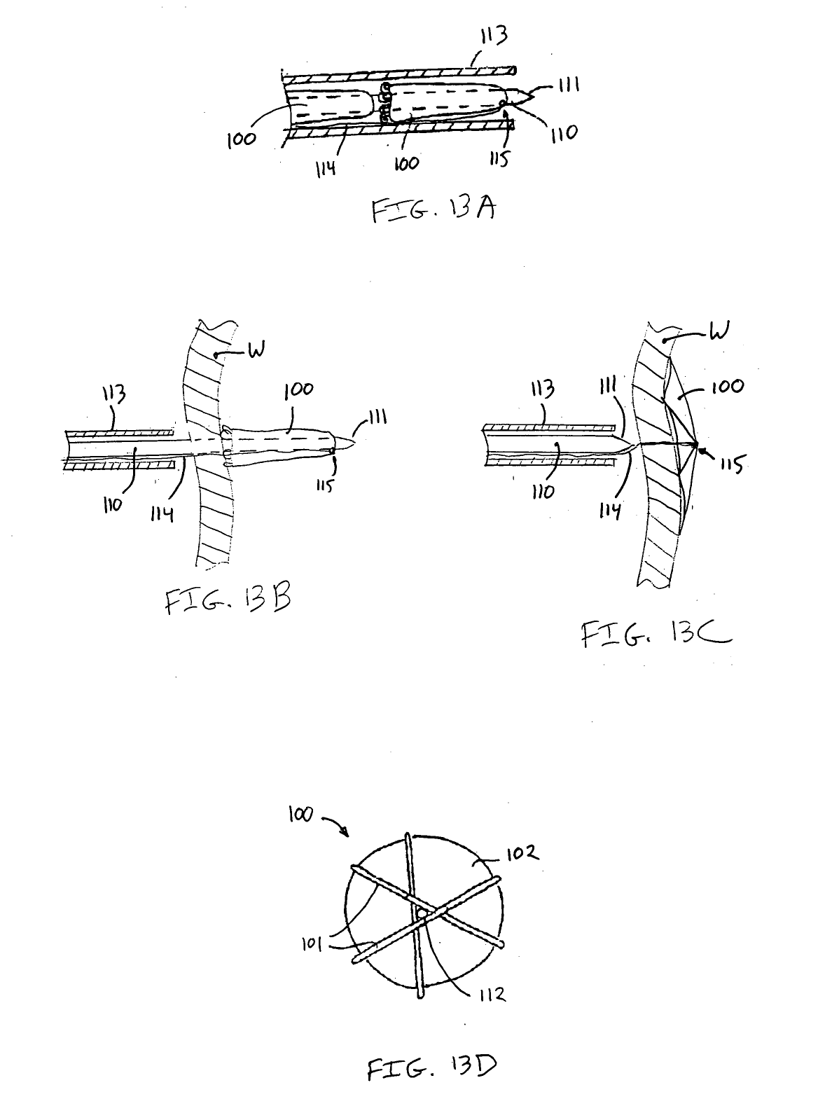

Referring to FIGS. 12A-12C, an alternative family of anchor embodiments is described, in which the anchors comprise self-expanding umbrellas 100. Each umbrella 100 comprises a plurality of support struts 101 and, optionally, membrane 102. Support struts 101 are preferably hinged to distal bushing 103, so that the struts may rotate from a reduced delivery profile within delivery catheter needle 16 (FIG. 12A) to an expanded deployed profile (FIGS. 12B and 12C). Suitable materials for the struts include engineering plastics and metal alloys, such as nitinol. optionally, the ends of the struts opposite distal bushing 103 may be twisted into small loops to form relatively atraumatic end points.

In FIG. 12B, umbrella anchor 100 includes optional shank 105. Shank 105 is attached to distal bushing 103 at one end and includes a fixture point, such as eyelet 106, at the other end. Eyelet 106 provides an attachment point one or more sutures 107. Alternatively, as depicted in FIG. 12C, eyelet 108 may be provided on the distal bushing.

In the reduced delivery profile, struts 101 and shank 105 are substantially parallel. When deployed, struts 101 rotate radially outward from the pivot point located at the distal bushing. Membrane 102, if present, prevents further outward rotation of struts 101. Distal bushing 103 may include a sharpened distal tip (FIG. 12C) to facilitate penetration of the tissue wall.

In some embodiments, opposing struts 101 of umbrella anchor 100 may be formed from a continuous length of wire and include a loop similar to that of spring wire malecot anchor 85 of FIG. 10. The loops increase the elasticity of the struts so that the struts may be more readily folded into the reduced delivery profile and expanded to the expanded deployed profile without plastic deformation. In addition, the loops advantageously provide a fixture point for attachment of one or more sutures.

Membrane 102 provides a greater surface area for contact with the tissue wall, which in turn decreases the stress transmitted to the tissue wall. Membrane 102 preferably comprises a pliable material with sufficient strength and resiliency to permit the umbrella anchor to readily expand and collapse. In addition, the membrane is preferably fluid impermeable and porous. Optionally, membrane 102 may include slots or perforations to promote tissue ingrowth. Suitable materials for membrane 102 include, but are not limited to, dacron, TEFLON, nylon, silastic, pericardium and silk. Preferably, membrane 102 is stretched and extended flat over the struts to promote and facilitate the self-expansion of the anchor. Alternatively, the membrane material may be fan-folded between the struts.

Referring to FIGS. 13A-13D, umbrella anchors 100 alternatively may be delivered using obturator 110 disposed for translation within a lumen of delivery catheter 113. As depicted in FIG. 13A, umbrella anchors 100 are disposed in the reduced delivery profile around the shaft of obturator 110. Obturator 110 may include sharpened distal tip 111 to facilitate penetration of tissue wall W. In addition, one or more sutures 114 are attached to the umbrella anchor at fixture point 115.

In FIG. 13B, obturator 110 is shown extended from delivery catheter 113 so that its distal tip and umbrella anchor 100 penetrate tissue wall W. With respect to FIG. 13C, once obturator 110 and anchor 100 pass through the tissue wall, the obturator is retracted. Anchor 100 then either self-expands or is induced to expand by applying tension to suture 114.

With respect to FIG. 13D, struts 101 may be arranged to form opening 112 that permits passage of distal tip 111 of obturator 110. If membrane 102 is included, it also may include an opening for distal tip 111. As will be appreciated by those of skill in the art, the mesh anchors of FIGS. 4 and 5 also may be easily modified to include openings in the bushings so as to be usable with an obturator without departing from the scope of the present invention.

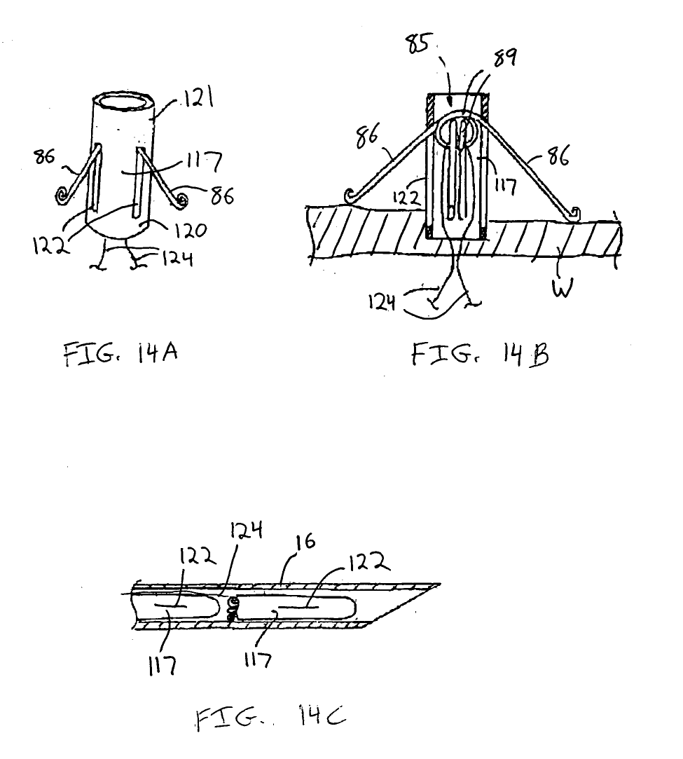

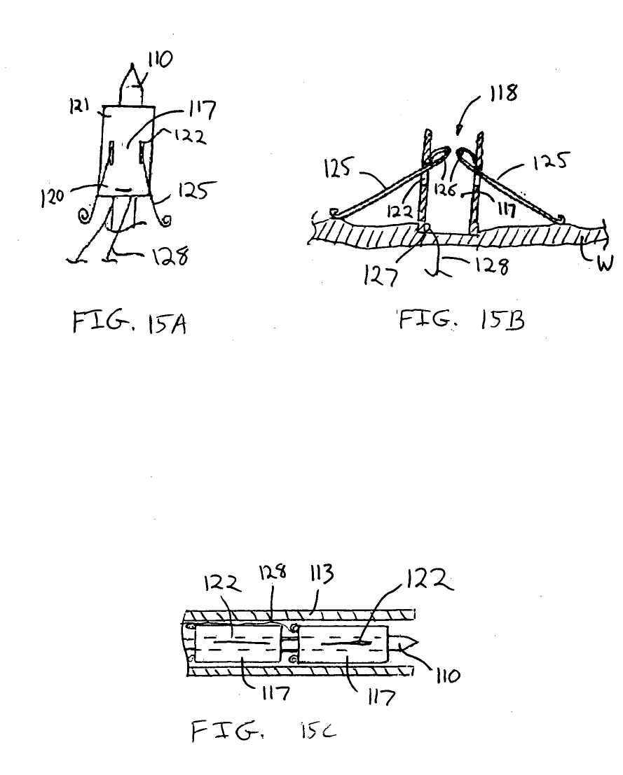

Referring now to FIGS. 14 and 15, an embodiment of an anchor disposed within slotted tube 117 is described. More particularly, FIGS. 14A-14C depict an expandable anchor, such as spring wire malecot anchor 85, disposed within slotted tube 117 and suitable for use with delivery catheter needle 16 of FIGS. 1-3. FIGS. 15A-15C likewise depict expandable anchor 118 disposed within slotted tube 117 and suitable for use with delivery catheter 113 (including obturator 110) of FIGS. 13A-13D.

Slotted tube 117 includes a central lumen, proximal bushing 120, distal bushing 121 and plurality of longitudinal slots 122 disposed between the bushings. Referring again to FIGS. 14A and 14B, in the expanded deployed profile, spring wire malecot anchor 85 is disposed partially within slotted tube 117, so that struts 86 protrude from slots 122. In the reduced delivery profile, struts 86 are disposed substantially within the tube (FIG. 14C). Loops 89 remain disposed within the tube in the expanded deployed configuration (FIG. 14B). The anchor optionally may include a membrane such as described with respect to FIG. 12.

Slotted tube 117 facilitates the alignment of struts 86 and augments the structural integrity of the anchor. In addition, slots 122 reduce the risk of anchor prolapse by providing stops that limit expansion of struts 86. Loops 89 may be disposed within the slotted tube via an interference fit, for example. Alternatively, loops 89 may be attached using methods such as welding, or may instead be disposed in a free-floating fashion within the lumen of slotted tube 117.

The anchor of FIG. 14 may be delivered using the delivery catheter of FIGS. 1-3. More particularly, as illustrated in FIG. 14C, a plurality of anchors including slotted tubes 117 and sutures 124 may be disposed sequentially within delivery catheter needle 16. Once the distal-most anchor is ejected from needle 16 by push rod 21, struts 86 will automatically self-expand. Tensioning the suture reinforces the expansion of the struts.

Alternatively, the anchors may be delivered using obturator 110 of FIG. 13B, wherein the loops are arranged to form an opening for the passage of the obturator. If the anchor includes membrane 102, the membrane should of course include an opening to facilitate passage of the obturator.

With respect to FIGS. 15A-15C, expandable anchor 118 is disposed within slotted tube 117 and suitable for use with the delivery catheter and obturator assembly of FIGS. 13A-13D. Anchor 118 comprises plurality of struts 125 having coil springs 126 disposed against an inner wall of slotted tube 117. In addition, anchor 118 includes a fixture point, such as eyelet 127, for attachment of one or more sutures 128.

When obturator 110 is inserted through slotted tube 117, it compresses the coil springs and forces the struts to rotate from the expanded deployed profile (FIG. 15B) to the reduced delivery profile (FIGS. 15A and 15C). Depending on the length of the slots, the struts may either be rotated against the outer surface of the slotted tube (i.e., if the slots are shorter than the struts as in FIG. 15A) or rotated within the lumen of tubular support (i.e., if the slots are longer than the struts). When obturator 100 is removed from anchor 118, the struts deflect radially outward.

Referring to FIGS. 16A-16B, anchor 130 comprising plurality of struts 132 and biasing element 133 is described. Preferably, the biasing element may be a spring that is pretensioned to bias the struts in an expanded deployed configuration in which the struts are substantially perpendicular to each other (FIG. 16A). In the reduced delivery configuration (FIG. 16B), the struts are substantially parallel within delivery catheter needle 16. Additionally, one or more sutures 135 may be coupled to a fixture point, such as eyelet 136, to facilitate approximation of the tissue walls.

Anchor 130 optionally includes membrane 137 attached to struts 132 to increase the contact area with the tissue wall in the expanded deployed configuration. Membrane 137 is similar to membrane 102 of FIG. 12, and preferably comprises a strong pliable material such as dacron, TEFLON, nylon, silastic, pericardium or silk. In addition, membrane 137 preferably is fluid impermeable and porous and may include slots or perforations to promote tissue ingrowth.

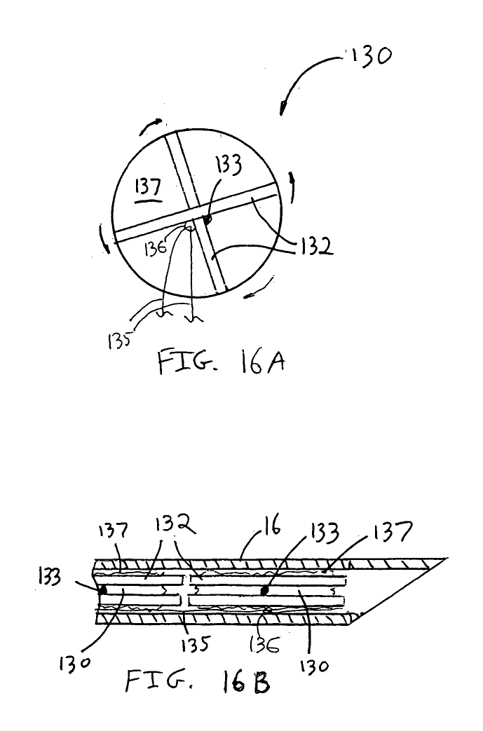

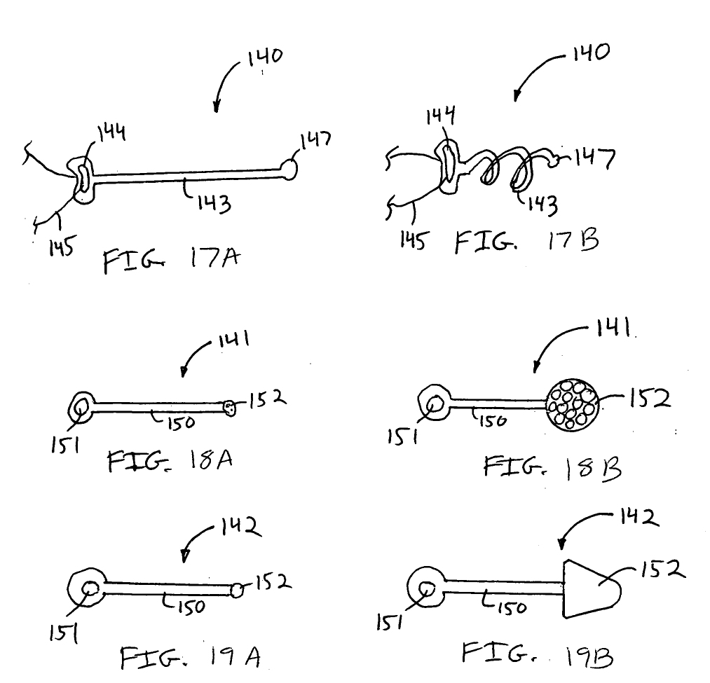

With respect to FIGS. 17-19, further anchor embodiments are described, including corkscrew anchors 140 (FIGS. 17A and 17B) and fluid expandable anchors 141 and 142 (FIGS. 18 and 19, respectively). Corkscrew anchor 140, shown in the reduced delivery profile in FIG. 17A, comprises elongate shaft 143 having a fixture point (e.g., eyelet 144) through which one or more sutures 145 may be threaded. As shown in FIG. 17B, the shaft assumes a coiled shape when deployed to the expanded deployed profile.

Elongate shaft 143 optionally may include sharpened distal tip 147 to facilitate penetration of tissue walls. If sharpened, distal tip 147 preferably comprises a bio-absorbable material, so that it will dissolve within the patient. If the shaft includes a blunt distal tip, delivery catheter 11 may include needle 16 (such as in FIGS. 1-3) to penetrate the tissue wall and deliver the anchor. Once the anchor is ejected from the delivery catheter, shaft 143 assumes the coiled shape as shown in FIG. 17B.

FIGS. 18 and 19 illustrate fluid expandable anchors 141 and 142, respectively, that comprise elongate shaft 150 having eyelet 151 at the proximal end and distensible fluid permeable enclosure 152 at the distal end. Enclosure 152 comprises an expandable core adapted to expand when contacted with fluids such as blood or water. The expandable core may be delivered in a solid granular state (FIG. 18) or as a solid material (FIG. 19). Suitable expandable core materials include polyvinylalcohol sponges such as the Hydofera PVA sponge (Hydrofera LLC, Williamantic, Conn.) and hydrogels such as polyacrylamide. The fluid expandable anchors may be delivered using the non-coring sharp-tipped delivery catheter 11 of FIGS. 1-3.

Referring now to FIGS. 20A and 20B, T-anchor 154 is described. T-anchor 154 comprises rod 155 and suture 156 attached approximately midway between the ends of rod 155. Suture 156 extends through hole 157 in the rod, but cannot be pulled through the hole because of stop 158 coupled to a distal end of suture 156. Alternatively, suture 156 may be attached using an eyelet or an adhesive. As shown in FIG. 20B, T-anchor 154 may be delivered using needle 16 and push rod 21 of the delivery catheter of FIGS. 1-3.

During delivery, the longitudinal axis of T-anchor 154 is substantially parallel to the longitudinal axis of the needle. However, once the T-anchor is ejected from the needle, it rotates approximately 90 degrees, so that the longitudinal axis of T-anchor 154 is substantially parallel to the tissue wall, thus reducing the risk that the T-anchor may be pulled through the tissue wall.

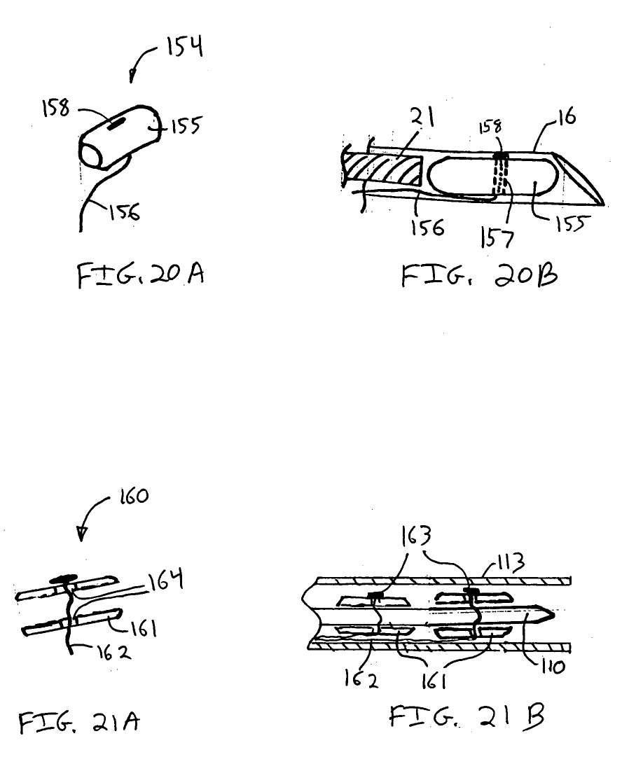

FIGS. 21A and 21B illustrate alternative T-anchor 160, which comprises tube 161 and suture 162 attached approximately midway between the ends of the tube. Like T-anchor 154, T-anchor 160 includes stop 163 that reduces the risk that suture 162 will be pulled through hole 164 in the tube. Of course the suture may also be attached using an eyelet or an adhesive. T-anchor 160 may be delivered using obturator 110 of the delivery system of FIG. 13.

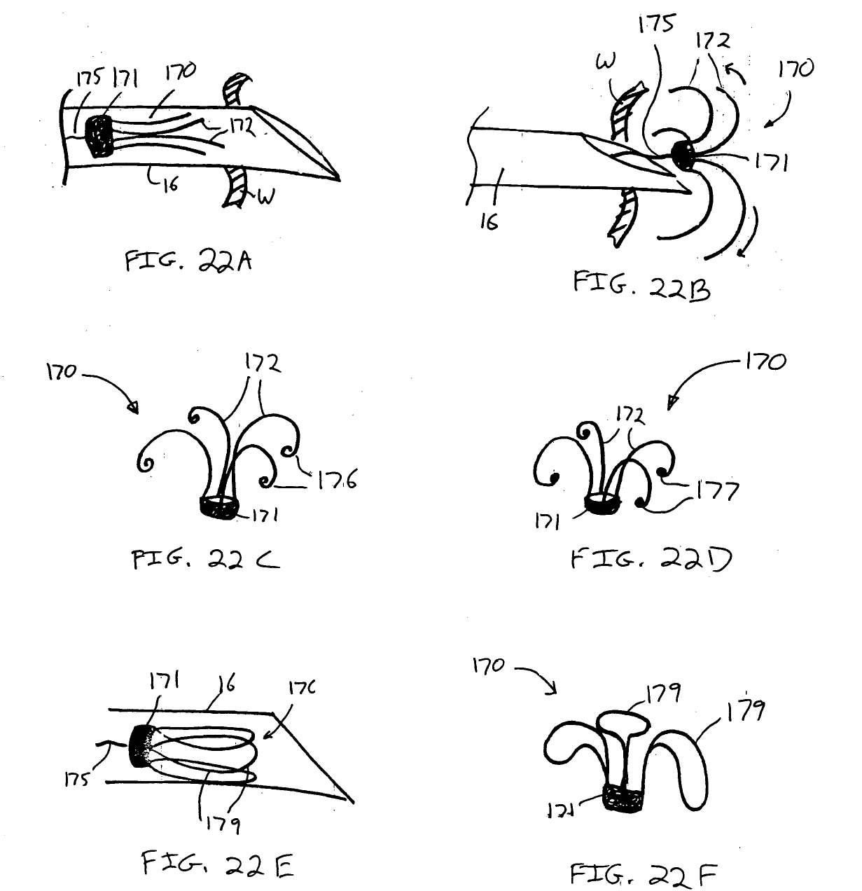

With respect to FIGS. 22A-22F, spider anchors 170 are described, and comprise hub 171 having plurality of wires 173 extending therefrom. Spider anchor also includes one or more sutures 175 coupled to hub 171. As depicted in FIG. 22A, spider anchor 170 may be loaded into delivery catheter needle 16 of FIG. 1 with the wires leading the hub, so that the interior surface of the needle retains the wires in a substantially straight reduced delivery profile.

In FIG. 22B, after needle 16 penetrates tissue wall W, spider anchor 170 is ejected from the needle and the wires assume a curved profile that prevents anchor 170 from being pulled back through the tissue wall. Advantageously, spider anchor 170 requires a minimal amount of clearance in front of the needle to properly deploy. Further, the anchor deploys in such a manner that expansion begins as soon as any portion of the anchor is free from the distal end of needle 16. The wires preferably are preshaped in the expanded curved profile.

With respect to FIG. 22C, the wires of spider anchor 170 may be made more atraumatic by forming the distal ends into coils 176 that capture the distal tips. Alternatively, as in the embodiment of FIG. 22D, the distal ends of wires may be formed into welded or molded atraumatic balls 177. In FIGS. 22E and 22F, respectively, a single wire is used to form atraumatic loops 179.

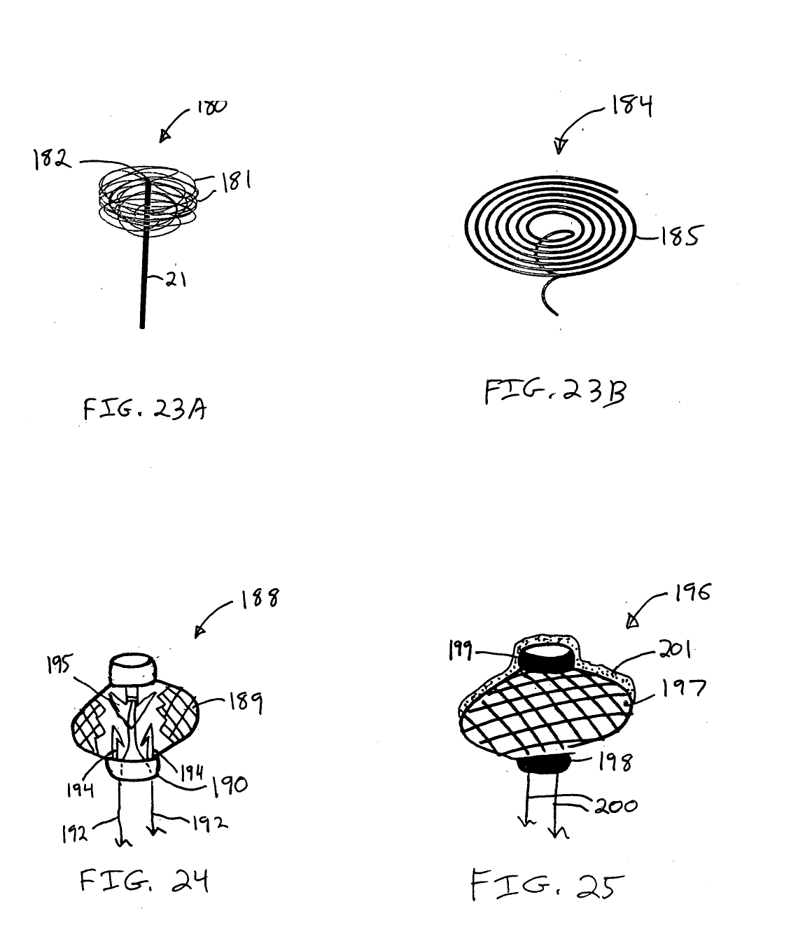

Referring to FIGS. 23A and 23B, additional alternative embodiments of expandable wire anchors constructed in accordance with the principles of the present invention are described. FIG. 23A shows wire anchor 180 formed from a plurality of wires 181 attached to hub 182. Alternatively, wire anchor 180 may be formed from a single piece of wire. FIG. 23B shows a disk-shaped wire anchor 184 formed from a single length of shaped wire 185.

In the expanded profile, wire anchors 180 and 184 may assume any of a variety of shapes, including substantially ball-shaped (FIG. 23A), disk-shaped (FIG. 23B), or randomly shaped. To deliver these expandable wire anchors, the wire is straightened and pushed through needle 16 using push rod 21 as disclosed with respect to FIGS. 1-3. When the wire exits the needle, it assumes its preformed expanded shape, as shown in FIGS. 23A and 23B.

With respect to FIG. 24, mesh anchor 188 (similar to mesh anchor 22 of FIGS. 4 and 5) is described. Mesh anchor 188 comprises braided sleeve 189, proximal bushing 190, distal bushing 191 and at least one suture 192. In addition, mesh anchor 188 includes an internal lock for retaining the anchor in its expanded shape. Internal lock preferably includes ferrule 194 and mating barb 195, which are adapted to engage and lock upon expansion of the anchor.

In operation, once the anchor is properly positioned relative to tissue wall W, the sutures are tensioned to pull the ferrule and barb into locking engagement. This locking feature retains the anchor in the expanded profile, even if tension on the suture is subsequently released. Of course, the internal locking feature may be incorporated into many of the other anchors described herein without departing from the scope of the present invention.

With respect to FIG. 25, mesh anchor 196 (similar to mesh anchor 22 of FIGS. 4 and 5) is described. Mesh anchor 196 comprises braided sleeve 197, proximal bushing 198, distal bushing 199 and at least one suture 200. In addition, distal bushing 199 and the distal half of braided sleeve 197 are coated with a bioactive agent 201. Bioactive agent 201 may be selected to promote tissue ingrowth and resultant adhesion of the anchor to adjacent organs. Alternatively, the bioactive agent may be selected to hinder tissue ingrowth and therefore reduce the possibility of adhesion to adjacent organs.

Although preferred illustrative embodiments of the present invention are described above, it will be evident to one skilled in the art that various changes and modifications may be made without departing from the invention. It is intended in the appended claims to cover all such changes and modifications that fall within the true spirit and scope of the invention.

What is claimed is:

1. An anchor for use with a gastric reduction system for narrowing a cross-sectional area of a gastro-intestinal lumen, the anchor comprising:
a sleeve including proximal and distal bushings;
wherein the sleeve is adapted to be reconfigured from a reduced delivery profile to an expanded deployed profile.

2. The anchor of claim 1, further comprising a suture coupled to the distal bushing and extending through the interior of the sleeve.

3. The anchor of claim 2, wherein the suture extends through an aperture in the proximal bushing.

4. The anchor of claim 3, wherein the sleeve is configured so that application of tension on the suture approximates the distal bushing to the proximal bushing.

5. The anchor of claim 3, wherein the sleeve is configured so that application of tension on the suture transitions the sleeve from the reduced delivery profile to the expanded deployed profile.

6. The anchor of claim 1, wherein the sleeve is braided.

7. The anchor of claim 1, wherein the sleeve is formed of individual monofilament elements.

8. The anchor of claim 7, wherein the monofilament elements are made of polyester, nylon, TEFLON, polypropylene or combinations of these materials.

9. The anchor of claim 1, wherein the sleeve comprises a shape memory material.

10. The anchor of claim 1, further comprising a filament attached to the proximal bushing to facilitate removal of the anchor.

11. The anchor of claim 1, further comprising an internal lock for retaining the sleeve in the expanded deployed profile.

12. The anchor of claim 1, wherein the internal lock comprises a ferrule and mating barb.

13. The anchor of claim 1, further comprising a coating of bioactive agent applied to an outer surface of the sleeve.

14. The anchor of claim 13, wherein the bioactive agent is selected to either promote or hinder tissue ingrowth.

15. The anchor of claim 1, wherein the sleeve comprises a plurality of longitudinal struts.

16. An anchor for use with a gastric reduction system, the anchor comprising:
a shank having proximal and distal ends; and
a reconfigurable member disposed on the distal end of the shank, the reconfigurable member having a reduced delivery profile and an expanded deployed profile.

17. The anchor of claim 16, wherein the proximal end of the shank defines an eyelet.

18. The anchor of claim 16, wherein the reconfigurable member comprises a plurality of struts affixed to the distal end of the shank, the plurality of struts having a reduced delivery profile wherein the plurality of struts are substantially parallel to the shank and an expanded deployed profile wherein the plurality of struts extend at angles away from the shank.

19. The anchor of claim 18, further comprising a membrane affixed to the plurality of struts.

20. The anchor of claim 18, wherein the shank is arranged so that tension applied to the shank urges the reconfigurable member to the expanded deployed profile.

21. The anchor of claim 16, wherein the reconfigurable member comprises a shape memory material.

22. The anchor of claim 16, wherein the reconfigurable member transitions from an elongate member in the reduced delivery profile to a corkscrew in the expanded deployed profile.

23. The anchor of claim 16, wherein the reconfigurable member transitions from an elongate member in the reduced delivery profile to a ball in the expanded deployed profile.

24. The anchor of claim 16, wherein the reconfigurable member transitions from an elongate member in the reduced delivery profile to a disk in the expanded deployed profile.

25. The anchor of claim 16, wherein the reconfigurable member is fluid inflatable.

26. An anchor for use with a gastric reduction system, the anchor comprising:
a plurality of struts, each one of the plurality of struts having a proximal end and a distal end, at least one of the proximal end or the distal end of each one of the plurality of struts affixed to a fixation point; and
a suture coupled to the fixation point,
wherein the plurality of struts have a reduced delivery profile wherein the plurality of struts are substantially parallel to one another and an expanded deployed profile where the plurality of struts extend at angles away from one another.

27. The anchor of claim 26, wherein the fixation point comprises a distal bushing and distal end of each one of the plurality of struts is coupled to the distal bushing and the proximal end of each one of the plurality of struts is coupled to a proximal bushing.

28. The anchor of claim 27, wherein the plurality of struts is formed by creating a plurality of through-wall longitudinal slots in a hollow cylinder.

29. The anchor of claim 28, wherein, in the expanded deployed profile, the plurality of struts bow radially outward to form a disk-like configuration.

30. The anchor of claim 26, wherein the fixation point comprises one or more loops that couple the distal ends of opposing ones of the plurality of struts, the loops acting as torsion springs to bias the anchor to the expanded deployed profile.

31. The anchor of claim 30, wherein each strut further comprises one or more flexure points.

32. The anchor of claim 27, wherein the plurality of struts form a petaled disk-like configuration is the expanded deployed profile.

33. The anchor of claim 26, wherein the plurality of struts are self-expanding.

34. The anchor of claim 33, further comprising a membrane affixed to the plurality of struts.

35. The anchor of claim 26, wherein the fixation point has a sharpened tip to facilitate tissue penetration.

36. The anchor of claim 36, wherein the struts are configured to form a central opening dimensioned to permit passage of an obturator therethrough.

37. The anchor of claim 30, wherein the plurality of struts are disposed within a tube including a plurality of slots, each one of the plurality of slots dimensioned to permit a corresponding one of the plurality of struts to extend therethrough.

38. The anchor of claim 37, wherein, in the reduced delivery profile, the plurality of struts are substantially disposed within the tube.

39. The anchor of claim 27, wherein the distal ends of the plurality of struts are disposed within a tube including a plurality of slots, each one of the plurality of slots dimensioned to permit a corresponding one of the plurality of struts to extend therethrough.

40. The anchor of claim 39, wherein the slotted tube is dimensioned to receive an obturator, insertion of the obturator causing the plurality of struts to rotate from the expanded deployed profile to the reduced delivery profile.

41. The anchor of claim 26, wherein the fixation point comprises a proximal hub and proximal end of each one of the plurality of struts is coupled to the proximal hub.

42. The anchor of claim 41, wherein the distal ends of adjacent ones of the plurality of struts are coupled together to form petals.

43. The anchor of claim 41, wherein the distal end of each of the plurality of struts terminates in an atraumatic ball.

44. An anchor for use with a gastric reduction system, the anchor comprising:
a suture having proximal and distal ends; and
a reconfigurable member disposed on the distal end of the suture, the reconfigurable member having a delivery profile and a deployed profile.

45. The anchor of claim 44 wherein the reconfigurable member comprises a tube.

46. The anchor of claim 45 wherein the tube comprises a plurality of through-wall slots, the reconfigurable member further comprising a plurality of struts, each one of the plurality of struts having a proximal end and a distal end, each one of the plurality of slots dimensioned to permit a corresponding one of the plurality of struts to extend therethrough.

47. The anchor of claim 44 wherein the reconfigurable member comprises first and second struts having longitudinal axes, the longitudinal axes of the first and second struts transitioning between the delivery profile, wherein the longitudinal axes are aligned, and the deployed profile, wherein the longitudinal axes intersect.

48. The anchor of claim 47, further comprising a membrane affixed to the first and second struts.

49. The anchor of claim 44, wherein the reconfigurable member comprises a tubular member having a longitudinal axis, the longitudinal axis of the tubular member transitioning between the delivery profile, wherein the longitudinal axis is aligned with a longitudinal axis of a delivery needle, and the deployed profile, wherein the longitudinal axis intersects the longitudinal axis of the delivery needle.
